(19) United States
(12) Patent Application Publication
(10) Pub. No.: US 2022/0010359 A1
(43) Pub. Date: Jan. 13, 2022

(54) DECISION TREE POLYMERASE CHAIN REACTION

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US)

(73) Assignee: Hewlett-Packard Development Company, L.P., Spring, TX (US)

(21) Appl. No.: 17/294,041

(22) PCT Filed: Dec. 14, 2018
(86) PCT No.: PCT/US2018/065758

§ 371 Date: May 14, 2021

(51) Int. Cl. C12Q 1/686

(52) U.S. Cl. CPC C12Q 1/686

(57) ABSTRACT

Examples relate to conducting Polymerase Chain Reactions (PCR). An example device for conducting PCR includes a primer applicator to add a first multi-primer set of a primer library to a first test chamber of a test chamber array. The example device also includes an amplification analyzer to determine if amplification has occurred in the first test chamber. The example device includes a device controller to instruct the primer applicator to apply a second multi-primer set of the primer library to the first test chamber in response to a detection by the amplification analyzer that amplification has not occurred in the first test chamber. Example devices instruct the primer applicator to apply a first primer subset of the first multi-primer set to a second test chamber of the test chamber array in response to a detection by the amplification analyzer that amplification occurred in the first test chamber.

### BACKGROUND

Polymerase chain reaction (PCR) is a method used in molecular biology to make multiple copies of a specific deoxyribonucleic acid (DNA) segment. Using PCR, a copy of a DNA sequence can be exponentially amplified to generate many more copies of that particular DNA segment. PCR employs two main reagents—primers and a DNA polymerase. In PCR, primers are specific to a particular DNA fragment to be amplified by the PCR process. If the DNA fragment corresponding to a primer is not present in a sample, then amplification through PCR may not occur.

### DESCRIPTION OF THE DRAWINGS

Certain examples are described in the following detailed description and in reference to the drawings, in which:

FIG. 1 is a block diagram of an example system for decision tree polymerase chain reactions;

FIG. 2 is a block diagram of an example cartridge with a bank of primers component over an amplification chip;

FIG. 3 is a block diagram of an example amplification chip with manual fill;

FIG. 4 is a block diagram of an example amplification chip with on-chip test chambers;

FIG. 5 is a flowchart of an example method for decision tree workflow enabling polymerase chain reactions;

FIG. 6 is a block diagram of an example schematic showing multiple chambers and decisions for decision tree polymerase chain reactions;

FIG. 7 is a block diagram of an example computing system for decision tree polymerase chain reactions;

FIG. 8 is a flowchart of an example method for decision tree polymerase chain reactions; and

FIG. 9 is a block diagram of an example non-transitory, computer-readable medium including instructions to direct a processor for decision tree polymerase chain reactions.

### DETAILED DESCRIPTION

This present disclosure relates to polymerase chain reactions (PCR). More specifically, techniques disclosed herein include a serial, decision tree based PCR reaction scheme. The reaction scheme disclosed can be implemented to obtaining a large amount of nucleic acid information on samples where nucleic acids are in limited supply. In an example, rapid PCR can be implemented with these techniques to obtain the net result in a relatively short amount of time in the order of minutes and hours compared to previous techniques providing results in an order of days or weeks.

This use of PCR can match a known primer or set of known primers to a target in a sample of DNA. The ability to generate a detectable amplification response through the introduction of primers indicates which known primers correspond to the samples. Accordingly, as the primers are known, the genotyping information of the sample can be identified. Using the techniques disclosed herein, a large amount of genotyping information from a relatively limited about of nucleic acid compared to previous methods. The more efficient use of limited amounts of nucleic acid or sample can reduce the number of test chambers needed and thus result in a smaller amplification chip footprint. The techniques described herein further reduce performing a full factorial interrogation of nucleic acid. An ancillary benefit of needing less sample starting material for PCR analysis is that there is less need to replicate a sample through pre-amplification steps and thus less chance for amplification bias. Full factorial analysis, unlike the presently disclosed techniques would demand a larger number of nucleic acids, large number of amplification tests, larger test chambers, larger amplification chips, and a longer time of analysis.

A few example genotyping analyses using limited nucleic acid samples includes the analysis of single cells, and rare cells, such as in detection and identification of sepsis, fetal cells, circulating tumor DNA, and circulating tumor cells. Issues of limited nucleic acid samples also can delay or increase expense for the analysis of circulating free DNA, endosomes. Small amounts of nucleic acid may also be the only sample available such as in small amount attempts from a patient including such as fingerpick analysis or trace sample from a crime scene. Other applications of these PCR techniques can include the construction of DNA-based phylogenies to identify organisms. These analyses may also assist in the functional analysis of genes, diagnosis and monitoring of hereditary diseases, amplification of ancient DNA, analysis of genetic fingerprints for DNA profiling in forensic science and parentage testing, and detection of pathogens in nucleic acid tests for the diagnosis of infectious diseases. Use of the decision tree methodology techniques described herein could increase the speed of these analyses while decreasing time for analysis as well as the need for a pre-amplified input sample or large amount of sample.

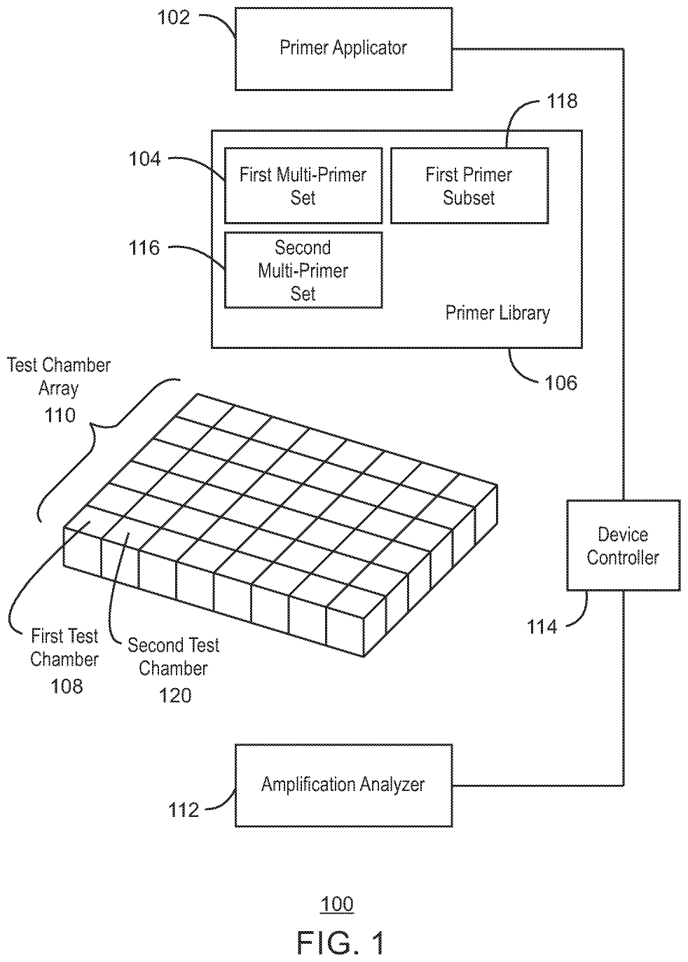

FIG. 1 is a block diagram of an example system for decision tree polymerase chain reactions 100. The decision tree polymerase chain reactions system can perform serial nucleic acid amplifications in order to identify unknown nucleic acid targets. These unknown targets can be identified even from a limited amount of nucleic acid in sample due to the redundancy permitted in the disclosed decision tree technique.

In an example, the system includes a primer applicator 102. The primer applicator 102 can be plastic, metal, or other similar rigid material and can dispense or apply primers to a target location by volume. In an example, the primer may be applied in a solution that is premixed or dry. The system 100 may have separated the sample nucleic acid into each of the chambers prior to starting or before each test chamber test is conducted. The primer applicator 102 may also dispense other liquids such as water before or after applying the primers. The primer applicator 102 may apply multiple-primers simultaneously based on how these primers are grouped together. For example, the primer applicator 102 may apply a first multi-primer set 104 from a primary library 106 to a first test chamber 108 of a test chamber array 110. In an example, the primer library is designed as to minimize primer interactions.

The first test chamber 108 may have included a sample that was preloaded or applied or inserted into the first test chamber 108 prior to the addition of a primer or multi-primer set. In an example, there are at least 10 copies of nucleic acid per chamber, to ensure that if the correct primer is loaded into the chamber, amplification will take place. This number of sample nucleic acid can be higher or lower. A specific primer or multi-primer set may interact with the sample or not based on the identity of the sample. If the identity of the sample in a test chamber corresponds to a primer applied to the test chamber, then amplification may occur.

The system 100 includes an amplification analyzer 112 to determine if amplification has occurred. The amplification analyzer 112 can include a fluorescence detector such as a microscope. A device controller 114 can use the reading of the amplification analyzer 112 to control the next action by the primer applicator 102. In an example, if the first multi-primer set 104 when added to the first test chamber 108 did not lead to amplification measured by the amplification analyzer 112, the primer applicator 102 next applies a second multi-primer set to the first test chamber 108. Reuse of the first test chamber 108 to test the amplification results on the same sample it contains reduces the amount of sample needed for experimentation.

However, if the amplification analyzer 112 detected amplification when the first multi-primer set is added to the first test chamber 108, then the device controller 114 instructs the primer applicator to apply the first primer subset 118. The first primer subset 118 is a smaller subset selected from the primers contained in the first multi-primer set 104. The primer applicator 102 applies the first primer subset 118 to the second test chamber 120. The amplification analyzer 112 then analyzes the second test chamber 120 to identify if amplification occurs. Based on these results, the device controller 114 can instruct the primer applicator 102 with the next course of action. This process is repeated until a decision tree showing which primers and primer sets, subsets, and supersets result in amplification. The decision tree can be corresponded to the known primers and primer sets added. This creates a decision tree of known successful amplification experiments which are able to identify the class, subclass, and sub subclasses to which the nucleic acid belongs to. In an example, knowing theses classes can correspond to the serotype or a particular organism such as a bacteria.

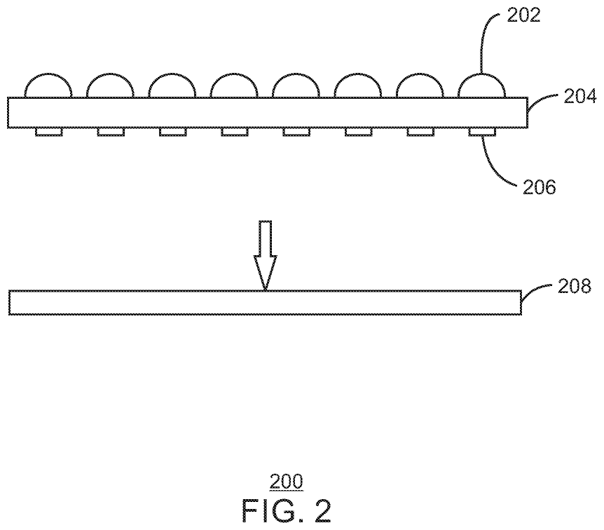

FIG. 2 is a block diagram of an example cartridge with a bank of primers component 200 over an amplification chip. The bank of primers component 200 can include reagent storage 202, support material 204, and ejection die 206.

As used herein, the primers may be stored in the reagent storage 202 shown in FIG. 2. The support material 204 may provide a holding structure for the reagent storage 202 and the ejection die 206. Additionally the support material 204 may be shaped to allow the easy handling of the cartridge into and out of a PCR machine. In some cases, primers to be added to a specific test chamber are shipped in dry form and reconstituted in buffer before reaction in a test chamber. Thus the reagent storage 202 may include a dry primer collection that can be reconstituted before dispensing through the ejection die 206. As shown in FIG. 2, the reagent storage may take the form of blister packs. In an example, the reconstituting buffer maybe added separately or may already be located in the reagent storage 202.

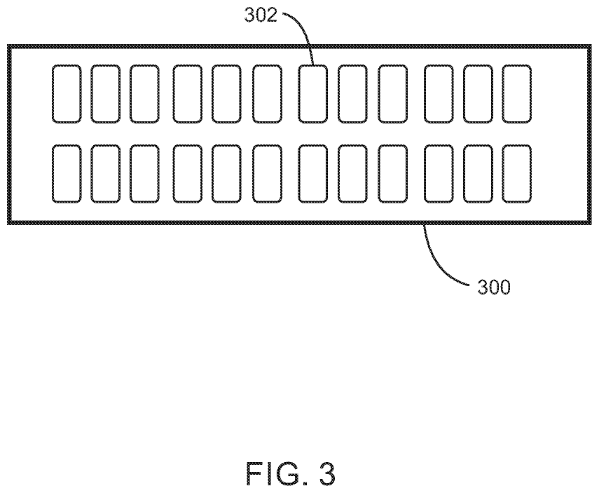

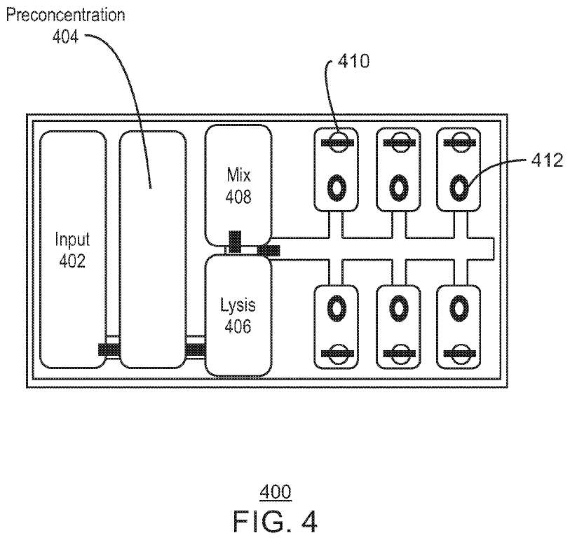

When operated, the bank of primers component 200 may eject the reagent through the ejection die into the amplification chip 208. The amplification chip 208 may take a variety of forms, two of which are show in FIG. 3 and FIG. 4 respectively. The amplification chip 208 can include the test chamber array 110 as shown in FIG. 1. The specific reagent storage 202 module may include a single primer, a multi-primer set, or a subset of primers. In an example, the grouping of primers in the reagent storage 202 is designed so that primers that amplify a variety of nucleotide sequences from the same organism or class of organism are grouped together. In an example, the grouping of primers in the reagent storage 202 is designed so that if multiple primers are stored in reagent storage 202 together, their reactions and chemical interactions with each other are minimal or absent.

FIG. 3 is a block diagram of an example amplification chip with manual fill 300. The view shown of this amplification chip with manual fill 300 is a top view facing down in the direction each test chamber 302 may be filled by a primer applicator 102. Each test chamber 302 of the amplification chip with manual fill may be relatively large openings sized to allow application or dispensing of fluid from multiple sources. These manual fill chambers enable the amplification chip to be able to be modified into a custom developed assay. In this example, a computing module may be able to group primers into a desired primer library so that the PCR analysis fills the order of the primer cartridges to best match the needs of the analysis.

In an example, a separate dispenser may dispense primers or primer mixes. In an example, a separate dispenser may dispense a solution of a PCR polymerase and a deoxyribonucleotide triphosphate (dNTP) to the first test chamber. Examples of PCR polymerases that can be used include polymerase are sold under the trademarks KAPPA 2G™, PHUSION™, or PHIRE™. Other preparation, pre-amplification, dilution, elution, or washing fluids may be added as well by either different dispensers or all through the same applicator.

FIG. 4 is a block diagram of an example amplification chip with on-chip test chambers 400. The amplification chip with on-chip test chambers 400 includes prefilled chambers to prepare a sample for testing and amplification with various primers or primer sets being tested. In an example, the amplification chip with on-chip test chambers 400 can include a sample input 402. In an example, there may be a single input for the entire amplification chip. In an example, there may be multiple inputs for the amplification chip. In the case where there is a single input, this is possible in part due to the interconnections between the input 402 and the other chambers of the amplification chip with on-chip test chambers 400.

The amplification chip with on-chip test chambers 400 may include a pre-concentration chamber 404 where a sample may be pre-concentrated using methods such as solvent extraction followed by solvent trapping, or sorbent trapping with subsequent solvent elution or thermal desorption. Other methods of pre-concentration of a sample may also be used as appropriate depending on the sample delivery medium. The amplification chip with on-chip test chambers 400 may include a lysis chamber 406 for cell lysis of an input sample. The amplification chip with on-chip test chambers 400 may include a mix chamber 408 may mix together the input sample with a solution of a PCR polymerase and a deoxyribonucleotide triphosphate (dNTP) or any other components needed for PCR to occur except for the primers. The amplification chip with on-chip test chambers 400 can include a number of chambers that a sample may be split between a number of chambers where individual primers may be applied. In an example, a primer solution may be applied through a thermal inject printing (TIJ) nozzles 410 in each of the chambers. The TIJ nozzles 410 may be. The amplification chip with on-chip test chambers 400 may also make use of an orifice 412 that can be sized for the scale of microfluidics of amplification on a chip. These orifices 412 may be used for amplification analysis, pressure relief, or addition of additional reagents. Using the sequence described at least in FIGS. 1, 5, 6, and 8, this amplification chip is enabled to allow a greater number of amplification tests for a set amount of sample. The redundant use of a chamber and the sample within enables additional amplification tests to be conducted per chamber. The design of this amplification chip can result correspond with factory filled cartridges of primers, e.g. the reagent storage 202 of FIG. 2 in the form of blister packs and other prefilled packs. For prefilled packs of reagent storage 202 there is less flexibility for the user, but also reduction of user error. In an example, amplification chips with on-chip testing can be implemented in example systems such as ink jet printers. In an example, printers may be adapted to be used can include printers available from HP Inc. under the trademark of HP® available from HP® Inc., a Delaware limited liability company in Texas 11445 Compaq Center Drive West Houston, Tex. 77070. Other printers, cartridges, primer dispensers, and test chamber arrays may be used implementing these techniques.

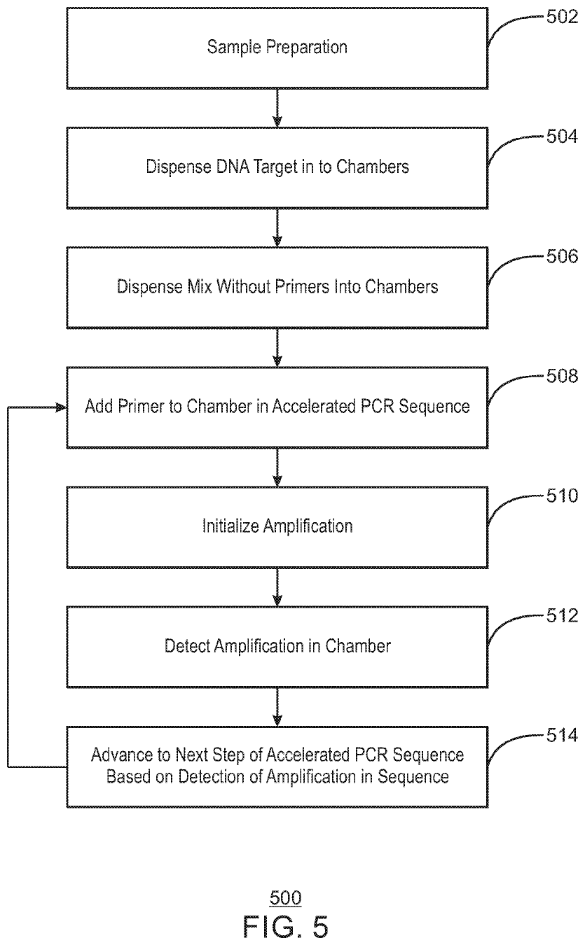

FIG. 5 is a flowchart of an example method for decision tree workflow 500 enabling polymerase chain reactions. The method for decision tree workflow 500 can make use of the systems, components, and hardware shown in FIG. 1-4 as well as variations of these devices.

At block 502, the method for decision tree workflow 500 includes sample preparation. This can vary depending on the way the sample is being provided to the test chambers. In the example of the amplification chip of FIG. 4, sample preparation can include insertion of the sample into the input 402 where the sample can then be pre-concentrated, any cells containing the sample can be lysed, and the nucleic acids may be absorbed, washed and eluted. In the example provided by the amplification chip configuration of FIG. 3, sample preparation as disclosed above can be conducted off of the amplification chip either within the PCR system or separately prior to being added to the test chambers.

At block 504, the prepared DNA targets are dispensed into the chambers. These DNA targets constitute the samples to be tested. The reuse of test chambers with no amplification detected after the addition of primers enables fewer chambers and thus less sample. The DNA dispensing may be manually done, automatically done by the PCR device, or inserted into an input on an amplification chip that disperses the DNA to be tested to each of the test chambers of the amplification chip.

At block 506, mix without the primers are dispensed into the test chambers. In an example, this step may overlap with dispensing the DNA target into the chambers. The mix can be a solution of a PCR polymerase and a deoxyribonucleotide triphosphate (dNTP) or any other components needed for PCR to occur except for the primers. The mix can be added manually, through a dedicated jet or nozzle for dispensing mix, or through an orifice or centralized mix input on the amplification chip.

At block 508, a primer can be added to a test chamber in an accelerated PCR sequence. In an example, the accelerated PCR sequence is responsive to detection of amplification. Accelerated PCR sequence also includes reuse of test chambers when a sample does not show an amplification result from a first primer added. In an accelerated PCR sequence a first step involves adding a first primer or multi-primer set to the first test chamber.

At block 510, once at least one primer has been added to at least the first test chamber, amplification can be initialized. In some examples, amplification may include a thermocycling step whereby a themocycling element repeatedly aids in denaturing the DNA and allowing the primers to potentially bind if the match a target, where replication of a target can occur as the test chamber cools. In some examples, the thermocycling can occur on a chip element, in other examples a thermocycling element may be applied separately. In an example, a thermocycling element can include a heater, thermoelectric cooler (TEC), air cooling, liquid cooling or other components that may controllably raise or lower the temperature in specified test chambers, for example the test chamber that is being tested or to which a primer was most recently added.

At block 512, an attempt to detect amplification in the chamber is made. In an example, an amplification detector can be based on florescence. In an example, an amplification detector can be a florescence microscope. At block 514 a compute or control module can use the results of an amplification detection in order to determine the next step of accelerated PCR sequence. In an example, if no amplification is detected, another primer or multi-primer set may be added to the same test chamber and redoing this process again from block 508. If an amplification detection is made, then a subset of the primers in a multi-primer set may be added to a newly prepared chamber that is tested with the added subset of the primers starting at step 508. In either case, this process may be iterated until all primers have been tried against the sample as they are grouped in multi-primer sets. Alternatively, the process may be iterated until the subset of primers that cause an amplification is equal to a single primer. In another example, the process may be iterated until a sufficient amplification profile is obtained based on which groups of primers led to amplification such that a desired amount of information about the identity and nature of the sample has been obtained.

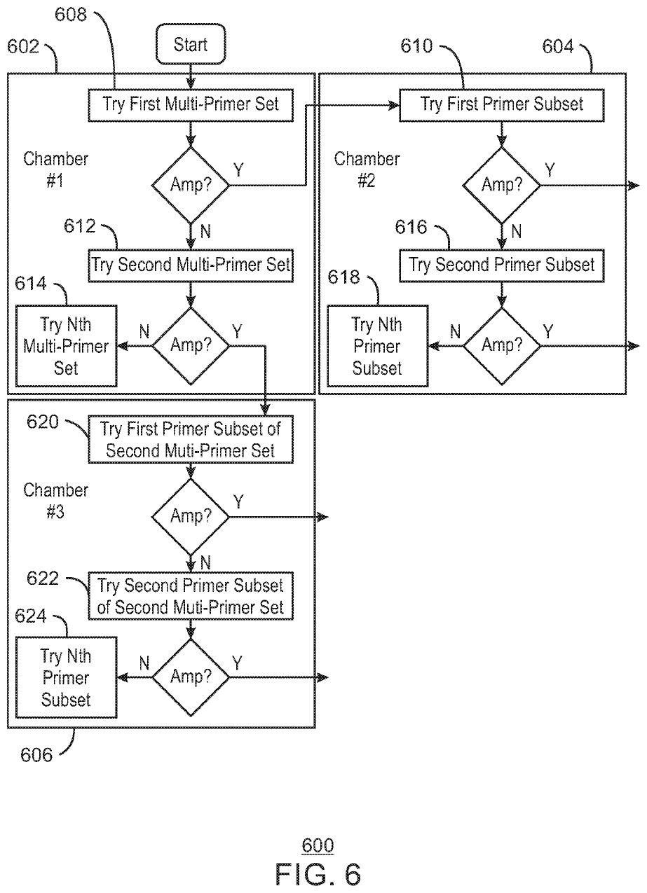

FIG. 6 is a block diagram of an example schematic showing multiple chambers and decisions for decision tree polymerase chain reactions 600. FIG. 6 only shows a few steps in what may be a more extensive, ongoing, and iterated process. Further, the layout of the various chambers are not intended to indicate a physical layout of the chambers in a device, but are instead shown arranged in the shown configuration for ease of concept explanation. For example, a first chamber, chamber #1 602, can be located next to chamber #2 604, with chamber #3 606 next to chamber #2 604 in a linear sequence. In another example, these chambers may not be near each other at all. The proximity of each chamber can be accommodated with sufficient logical labeling to better understand the results and steps of this accelerated PCR amplification. In each chamber in FIG. 6, it is assumed that sufficient sample material of DNA are present and prepared for additions of a primer to attempt amplification.

In FIG. 6, the PCR analysis begins in chamber #1 602, where a first multi-primer set is added to or tried in 608 chamber #1 602. After thermocycling the first multi-primer set with the sample, a determination is made about whether or not amplification is detected in chamber #1 602. If yes, amplification has been detected, then it can safely be assumed that at least of the primers within the first multi-primer set caused the amplification. Accordingly, testing can begin with a smaller subset in a new chamber so that amplification isn't falsely detected by reusing the already amplified sample in chamber 41. In response to an amplification being detected in chamber #1 from the addition of the first multi-primer set, the PCR analysis tries a first primer subset 610 applied to chamber #2.

If instead, after trying the first multi-primer set 608 in chamber #1 602 there is no detected amplification, the second multi-primer set can be tried 612 in is tried in chamber #1 602. In this example, the primers in the first multi-primer set are not in the second multi-primer set. Accordingly, if a detection of amplification is found after addition of the second multi-primer set when no amplification was detected after addition of the first multi-primer set, then the amplification detected was a result of at least one primer in the second multi-primer set. However, if no amplification response is detected after trying the second multi-primer set 612, then another multi-primer set, e.g. the Nth multi-primer set may be tried 614, where N is the number of iterations of amplification that have been attempted in chamber #1 without a detection of amplification. In an example, this iterating can proceed either until there are no remaining multi-primer groups or until a detection of amplification is found. Each multi-primer set may be distinct from every other multi-primer set. In an example, this can mean that if a primer is in one multi-primer set, then that primer is not in any other multi-primer set.

Returning to the circumstance where an amplification detection has been made in the first multi-primer set and now a first primer subset is being tested against a fresh sample in chamber #2 604. Once the first primer subset has been tried 610 in chamber #2 604 an amplification detection can be attempted. If amplification is detected, then this process may be repeated with an even smaller subset taken from the first primer subset. This process may iterate until sufficient amplification data has been gathered and correlated to the types of primers causing the amplification response. This process of testing smaller and smaller amplified subsets may also stop if only a single primer is added to a chamber to test if it, when added, results in an amplification. As before, any test in chamber #2 that results in amplification either continues tests of a smaller group in a new test chamber, or concludes the testing.

If however, there is no amplification response when the first primer subset is tried 610, then a second primer subset can be tried 616. A second primer subset is a subset distinct from the first primer subset while still belonging to the first multi-primer set. In an example, no primer in the second primer subset is present in the first primer subset. If the second primer subset has been tried 616 in chamber #2 604 and amplification is detected, then this process may be repeated with an even smaller subset taken from the second primer subset. As before, this process may iterate until sufficient amplification data has been gathered and correlated to the types of primers causing the amplification response. If the second primer subset has been tried 616 in chamber #2 604 and amplification is not detected, then this process may be repeated with other primer subsets of the first multi-primer subset 618. Indeed, an Nth primer subset may be tried iteratively for amplification, where the N represents the number of attempts of primer subsets that have been added to chamber #2 604 without amplification results.

Returning to the circumstance where an amplification detection has been made in response to a second multi-primer set being tried 612 and now a second primer subset is being tested 620 against a fresh sample in chamber #3 606. Once the first primer subset of the second multi-primer set has been tried 620 in chamber #3 606 an amplification detection can be attempted. If amplification is detected, then this process may be repeated with an even smaller subset taken from the first primer subset of the second multi-primer set. This process may iterate until sufficient amplification data has been gathered and correlated to the types of primers causing the amplification response. This process of testing smaller and smaller amplified subsets may also stop if the subset size is a single primer and results in an amplification detection. As before, any test in chamber #3 606 that results in amplification either continues tests of a smaller collection of primers in a new test chamber, or concludes the testing.

If however, there is no amplification response when the first primer subset of the second multi-primer set is tried 620, then a second primer subset of the second multi--primer subset can be tried 622. A second primer subset of the second multi-primer set is a subset distinct from the first primer subset of the second multi-primer subset while still belonging to the second multi-primer set. In an example, no primer in the second primer subset of the second multi-primer subset is present in the first primer subset of the second multi-primer subset. If the second primer subset of the second multi-primer subset has been tried 622 in chamber #3 606 and amplification is detected, then this process may be repeated with an even smaller subset taken from the second primer subset of the second multi-primer subset. As before, this process may iterate until sufficient amplification data has been gathered and correlated to the types of primers causing the amplification response. If the second primer subset of the second multi-primer subset has been tried 622 in chamber #3 606 and amplification is not detected, then this process may be repeated with other primer subsets of the second multi-primer subset 624. Indeed, an Nth primer subset may be tried iteratively for amplification, where the N represents the number of attempts of primer subsets that have been added to chamber #3 606 without amplification results.

As a note, if in chamber #1 602, an amplification result is detected after trying the first multi-primer subset, this does not rule out, in some example, the process returning to make attempts with other primers other than those in the first multi-primer set. The determination of if this type of testing is desired can be programed and decided for test chamber arrays on manual fill amplification chips. Testing thoroughness can also be based on the size and number of sample chambers present in pre-made amplification chips, such as those seen in FIG. 4.

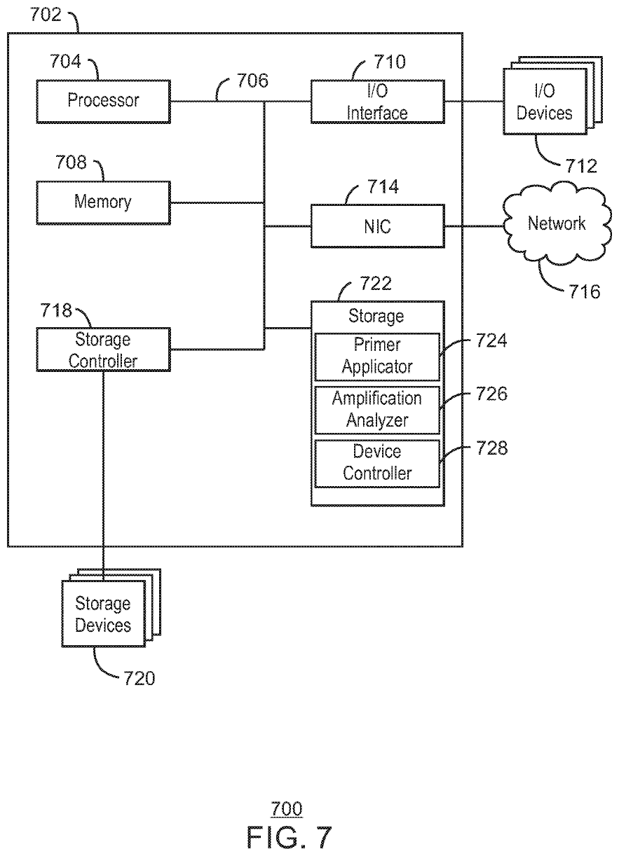

FIG. 7 is a block diagram of an example computing system for decision tree polymerase chain reactions. The system for decision tree polymerase chain reactions can be a standalone computing device 702 for dispensing primers and potentially other PCR components. This computing device 702 can include a desktop computer, laptop, tablet, mobile phone, smart device, printer hub, printer controller, or other computing devices. The system 700 for decision tree polymerase chain reactions includes at least one processor 704. The processor 704 can be a single core processor, a multicore processor, a processor cluster, and the like. The processor 704 can may include a graphics processing unit (GPU), an application specific integrated circuit (ASIC), or a field programmable gate array (FPGA), or any combination thereof to implement video processing. The processor 704 can be coupled to other units through a bus 706. The bus 706 can include peripheral component interconnect (PCI) or peripheral component interconnect express (PCIe) interconnects, Peripheral Component Interconnect eXtended (PCIx), or any number of other suitable technologies for transmitting information.

The computing device 702 can be linked through the bus 706 to a memory 708. The system memory 708 can include random access memory (RAM), including volatile memory such as static random-access memory (SRAM) and dynamic random-access memory (DRAM). The system memory 708 can include directly addressable non-volatile memory, such as resistive random-access memory (RRAM), phase-change memory (PCRAM), Memristor, Magnetoresistive random-access memory, (MRAM), Spin-transfer torque Random Access Memory (STTRAM), and any other suitable memory that can be used to provide computers with persistent memory.

The processor 704 may be coupled through the bus 706 to an input/output (I/O) interface 710. The I/O interface 710 may be coupled to any suitable type of I/O devices 712, including input devices, such as a mouse, touch screen, keyboard, display, VR/AR controllers through body movement detection cameras, handheld controllers and the like. The I/O devices 712 may be output devices such as a display, VR/AR goggles, a projector, and the like.

The computing device 702 can include a network interface controller (NIC) 714, for connecting the computing device 702 to a network 716. In some examples, the network 716 can be an enterprise server network, a storage area network (SAN), a local area network (LAN), a wide-area network (WAN), or the Internet, for example. The processor 704 can be coupled to a storage controller 718, which may be coupled to one or more storage devices 720, such as a storage disk, a solid state drive, an array of storage disks, or a network attached storage appliance, among others.

The computing device 702 can include a non-transitory, computer-readable storage media, such as a storage 722 for the long-term storage of data, including the operating system programs and user file data. The storage 722 can include local storage in a hard disk or other non-volatile storage elements. While generally system information may be stored on the storage 722, in this computing device 702, the program data can be stored in the memory 708. The storage 722 may store instructions that may be executed by the processor 704 to perform a task.

The storage 722 can include a primer applicator 724 to add a first multi-primer set of a primer library to a first test chamber of a test chamber array. In an example, the test chamber array is a single chip. The first multi-primer set may include a first primer and the second multi-primer set comprises a second primer distinct from the first primer. In this example, the first primer and second primer are nucleotide sequences from different bacterial phyla. The primer library may contain a known number of multi-primer sets, the first test chamber comprises a first test chamber volume, and wherein the primer applicator 724 applies the first multi-primer set to the first test chamber in an aliquot smaller than the first test chamber volume divided by the known number of multi-primer sets.

As used herein, the primer library can be stored in a blister pack prior to application by the primer applicator 724. Further, it is noted that primers can be shipped in dry form and reconstituted in buffer before reaction. In the context of this disclosure and the claims, the use of the term primer library refers to the groupings of primers included in each dispensing, where the groupings of primers share characteristics or map to the same class or group of organism or organ. To this end, the term primer library may include not only these separately grouped primers in a dry library but also the reconstituting buffer mixed together with these primers, or any other preparation and delivery components for these primers. To that end, in some examples, the primer applicator 724 adds a solution of a PCR polymerase and a deoxyribonucleotide triphosphate (dNTP) to the first test chamber. An example of PCR polymerases that can be used include polymerase that are sold under the trademark KAPA2G™ available from Kapa Biosystems, a Foreign Corporation registered in Massachusetts as a division of Roche Company headquartered in Basel, Switzerland. Another example of PCR polymerases that can be used include polymerase that are sold under the trademark PHUSION™ or PHIRE™ available from Thermo Fischer Scientific® Inc. headquartered in Waltham, Mass. These and other PCR polymerases are addable by the primer applicator 724.

The storage 722 can include an amplification analyzer 726 to determine if amplification has occurred in the first test chamber. In an example, the amplification analyzer 726 comprises a fluorescence detector. In another example, the amplification analyzer 726 recalibrates before measuring amplification in the first test chamber in response to the primer applicator 724 applying the second multi-primer set of the primer library to the first test chamber.

The storage 722 can include a device controller 728 to instruct the primer applicator 724 to apply a second multi-primer set of the primer library to the first test chamber in response to a detection by the amplification analyzer 726 that amplification has not occurred in the first test chamber. The device controller 728 may instead instruct the primer applicator 724 to apply a first primer subset of the first multi-primer set to a second test chamber of the test chamber array in response to a detection by the amplification analyzer 726 that amplification occurred in the first test chamber. In an example, the device controller 728 records that a test chamber of the test chamber array is determined to show amplification, where the device controller 728 to also record the identity of the mixture last added to the test chamber by the primer applicator 724. In the context of these recordings, this example shows that the mixture identified includes at least one of a primer, a plurality of primers, a plurality of multi-primer sets, or a plurality of multi-primer subsets.

It is to be understood that the block diagram of FIG. 7 is not intended to indicate that the computing device 702 is to include all of the components shown in FIG. 7. Rather, the computing device 702 can include fewer or additional components not illustrated in FIG. 7.

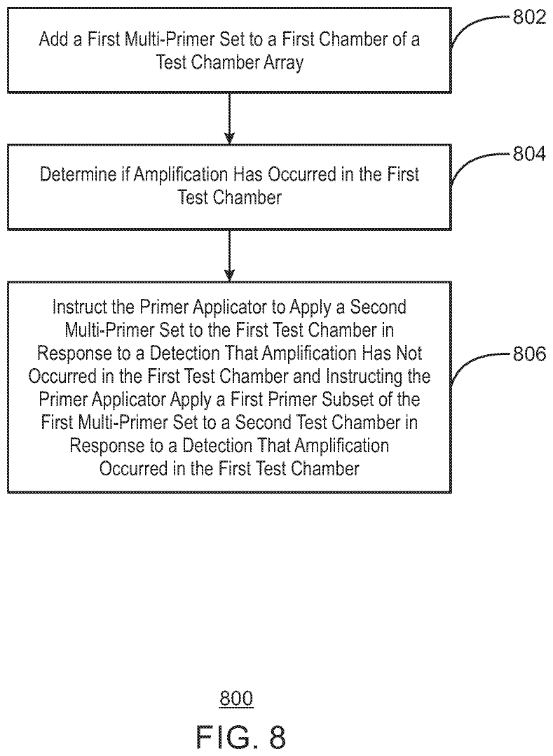

FIG. 8 is a flowchart of an example method for decision tree polymerase chain reactions. At block 802, a primer applicator can add a first multi-primer set of a primer library to a first test chamber of a test chamber array. In an example, the test chamber array is a single chip. The first multi-primer set may include a first primer and the second multi-primer set comprises a second primer distinct from the first primer. In this example, the first primer and second primer are nucleotide sequences from different bacterial phyla. The primer library may contain a known number of multi-primer sets, the first test chamber comprises a first test chamber volume, and wherein the primer applicator applies the first multi-primer set to the first test chamber in an aliquot smaller than the first test chamber volume divided by the known number of multi-primer sets.

As used herein, the primer library can be stored in a blister pack prior to application by the primer applicator. Further, it is noted that primers can be shipped in dry form and reconstituted in buffer before reaction. In the context of this disclosure and the claims, the use of the term primer library refers to the groupings of primers included in each dispensing, where the groupings of primers share characteristics or map to the same class or group of organism or organ. To this end, the term primer library may include not only these separately grouped primers in a dry library but also the reconstituting buffer mixed together with these primers, or any other preparation and delivery components for these primers. To that end, in some examples, the primer applicator adds a solution of a PCR polymerase and a deoxyribonucleotide triphosphate (dNTP) to the first test chamber. Examples of PCR polymerases that can be used include polymerase are sold under the trademarks KAPPA 2G™, PHUSION™, or PHIRE™. These and other PCR polymerases are addable by the primer applicator.

At block 804, an amplification analyzer can determine if amplification has occurred in the first test chamber. In an example, the amplification analyzer comprises a fluorescence detector. In another example, the amplification analyzer recalibrates before measuring amplification in the first test chamber in response to the primer applicator applying the second multi-primer set of the primer library to the first test chamber.

At block 806, a device controller to instruct the primer applicator to apply a second multi-primer set of the primer library to the first test chamber in response to a detection by the amplification analyzer that amplification has not occurred in the first test chamber. Within block 806, the device controller may instead instruct the primer applicator to apply a first primer subset of the first multi-primer set to a second test chamber of the test chamber array in response to a detection by the amplification analyzer that amplification occurred in the first test chamber. In an example, the device controller records that a test chamber of the test chamber array is determined to show amplification, where the device controller to also record the identity of the mixture last added to the test chamber by the primer applicator. In the context of these recordings, this example shows that the mixture identified includes at least one of a primer, a plurality of primers, a plurality of multi-primer sets, or a plurality of multi-primer subsets.

It is to be understood that the block diagram of FIG. 8 is not intended to indicate that the method 800 is to include all of the actions shown in FIG. 8. Rather, the method 800 can include fewer or additional components not illustrated in FIG. 8.

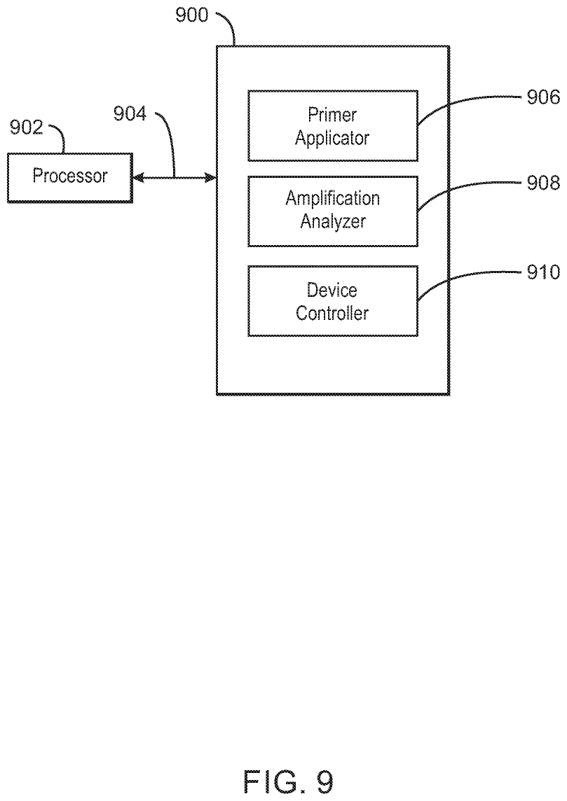

FIG. 9 is a block diagram of an example non-transitory, computer-readable medium including instructions to direct a processor for decision tree polymerase chain reactions. The computer readable medium 900 may include the storage 722 or the memory 708 of FIG. 7 and other suitable formats readable by the computing device. The computer readable medium 900 can include the processor 902 to execute instructions received from the computer-readable medium 900. Instructions can be stored in the computer-readable medium 900. These instructions can direct the processor 902 for decision tree polymerase chain reactions. Instructions can be communicated over a bus 904 as electrical signals, light signals, or any other suitable means of communication for transmission of data in a similar computing environment.

The computer-readable medium 900 includes a primer applicator 906 to add a first multi-primer set of a primer library to a first test chamber of a test chamber array. In an example, the test chamber array is a single chip. The first multi-primer set may include a first primer and the second multi-primer set comprises a second primer distinct from the first primer. In this example, the first primer and second primer are nucleotide sequences from different bacterial phyla. The primer library may contain a known number of multi-primer sets, the first test chamber comprises a first test chamber volume, and wherein the primer applicator 906 applies the first multi-primer set to the first test chamber in an aliquot smaller than the first test chamber volume divided by the known number of multi-primer sets.

As used herein, the primer library can be stored in a blister pack prior to application by the primer applicator 906. Further, it is noted that primers can be shipped in dry form and reconstituted in buffer before reaction. In the context of this disclosure and the claims, the use of the term primer library refers to the groupings of primers included in each dispensing, where the groupings of primers share characteristics or map to the same class or group of organism or organ. To this end, the term primer library may include not only these separately grouped primers in a dry library but also the reconstituting buffer mixed together with these primers, or any other preparation and delivery components for these primers. To that end, in some examples, the primer applicator 906 adds a solution of a PCR polymerase and a deoxyribonucleotide triphosphate (dNTP) to the first test chamber. Examples of PCR polymerases that can be used include polymerase are sold under the trademarks KAPPA 2G™, PHUSION™or PHIRE™. These and other PCR polymerases are addable by the primer applicator 906.

The computer-readable medium 900 includes an amplification analyzer 908 to determine if amplification has occurred in the first test chamber. In an example, the amplification analyzer 908 comprises a fluorescence detector. In another example, the amplification analyzer 908 recalibrates before measuring amplification in the first test chamber in response to the primer applicator 906 applying the second multi-primer set of the primer library to the first test chamber.

The computer-readable medium 900 includes a device controller 910 to instruct the primer applicator 906 to apply a second multi-primer set of the primer library to the first test chamber in response to a detection by the amplification analyzer 908 that amplification has not occurred in the first test chamber. The device controller 910 may instead instruct the primer applicator 906 to apply a first primer subset of the first multi-primer set to a second test chamber of the test chamber array in response to a detection by the amplification analyzer 908 that amplification occurred in the first test chamber. In an example, the device controller 910 records that a test chamber of the test chamber array is determined to show amplification, where the device controller 910 to also record the identity of the mixture last added to the test chamber by the primer applicator 906. In the context of these recordings, this example shows that the mixture identified includes at least one of a primer, a plurality of primers, a plurality of multi-primer sets, or a plurality of multi-primer subsets.

It is to be understood that the block diagram of FIG. 9 is not intended to indicate that the computer-readable medium 900 is to include all of the components shown in FIG. 9. Rather, the computer-readable medium 900 can include fewer or additional components not illustrated in FIG. 9.

While the present techniques may be susceptible to various modifications and alternative forms, the techniques discussed above have been shown by way of example. It is to be understood that the technique is not intended to be limited to the particular examples disclosed herein. Indeed, the present techniques include all alternatives, modifications, and equivalents falling within the scope of the following claims.

What is claimed is:

1. A molecule dispensing device for conducting Polymerase Chain Reaction (PCR):
a primer applicator to add a first multi-primer set of a primer library to a first test chamber of a test chamber array;
an amplification analyzer to determine if amplification has occurred in the first test chamber; and
a device controller to instruct the primer applicator to apply a second multi-primer set of the primer library to the first test chamber in response to a detection by the amplification analyzer that amplification has not occurred in the first test chamber, and wherein the device controller is to instruct the primer applicator to apply a first primer subset of the first multi-primer set to a second test chamber of the test chamber array in response to a detection by the amplification analyzer that amplification occurred in the first test chamber.

2. The molecule dispensing device of claim 1, wherein the first multi-primer set comprises a first primer and the second multi-primer set comprises a second primer distinct from the first primer.

3. The molecule dispensing device of claim 2, wherein the first primer and second primer are nucleotide sequences from different bacterial phyla.

4. The molecule dispensing device of claim 1, wherein the device controller records that a test chamber of the test chamber array is determined to show amplification, the device controller to also record the identity of the mixture last added to the test chamber by the primer applicator, wherein the mixture includes at least one of a primer, a plurality of primers, a plurality of multi-primer sets, or a plurality of multi-primer subsets.

5. The molecule dispensing device of claim 1, wherein the primer library is stored in a blister pack prior to application by the primer applicator.

6. The molecule dispensing device of claim 1, wherein the amplification analyzer comprises a fluorescence detector.

7. The molecule dispensing device of claim 1, wherein the amplification analyzer recalibrates before measuring amplification in the first test chamber in response to the primer applicator applying the second multi-primer set of the primer library to the first test chamber.

8. The molecule dispensing device of claim 1, wherein the primer library contains a known number of multi-primer sets, the first test chamber comprises a first test chamber volume, and wherein the primer applicator applies the first multi-primer set to the first test chamber in an aliquot smaller than the first test chamber volume divided by the known number of multi-primer sets.

9. The molecule dispensing device of claim 1, wherein the primer applicator adds a solution of a PCR polymerase and a deoxyribonucleotide triphosphate (dNTP) to the first test chamber.

10. The molecule dispensing device of claim 1, wherein the test chamber array is a single chip.

11. A method for conducting Polymerase Chain Reactions (PCR) comprising:
adding, with a primer applicator, a first multi-primer set of a primer library to a first test chamber of a test chamber array;
determining, with an amplification analyzer, if amplification has occurred in the first test chamber; and
instructing, with a device controller, the primer applicator to apply a second multi-primer set of the primer library to the first test chamber in response to a detection by the amplification analyzer that amplification has not occurred in the first test chamber.

12. The method for conducting PCR of claim 11, comprising adding a solution of a PCR polymerase and a deoxyribonucleotide triphosphate (dNTP) to the first test chamber with the primer applicator.

13. The method for conducting PCR of claim 11, comprising recording, with the device controller, that a test chamber of the test chamber array is determined to show amplification along with the identity of the mixture last added to the test chamber by the primer applicator, wherein the mixture includes at least one of a primer, a plurality of primers, a plurality of multi-primer sets, or a plurality of multi-primer subsets.

14. A computer readable medium for conducting Polymerase Chain Reaction comprising instructions that when executed on a processor instruct the processor to:
add, with a primer applicator, a first multi-primer set of a primer library to a first test chamber of a test chamber array;
determine, with an amplification analyzer, if amplification has occurred in the first test chamber;
instruct the primer applicator to apply a second multi-primer set of the primer library to the first test chamber in response to a detection by the amplification analyzer that amplification has not occurred in the first test chamber.

15. The computer readable medium of claim 14, wherein the instructions, when executed on the processor, instruct the primer applicator to add a solution of a PCR polymerase and a deoxyribonucleotide triphosphate (dNTP) to the first test chamber.
